(19) United States
(12) Patent Application Publication
(10) Pub. No.: US 2017/0020373 A1
(43) Pub. Date: Jan. 26, 2017

(54) METHOD AND APPARATUS FOR ENDOSCOPE STIFFENING

(71) Applicant: Risen Star Industries, LLC, McAllen, TX (US)

(72) Inventors: Jose Rodriguez-Sanjuan, McAllen, TX (US); Lucas Carlos Rodriguez, Dallas, TX (US)

(21) Appl. No.: 15/285,259

(22) Filed: Oct. 4, 2016

Related U.S. Application Data
(63) Continuation of application No. 14/293,779, filed on Jun. 2, 2014.

(51) Int. Cl. A61B 1/01; A61B 1/015; A61B 1/018; A61B 1/00

(52) U.S. Cl. CPC A61B 1/01; A61B 1/00066; A61B 1/015; A61B 1/018; A61B 1/00128; A61B 1/00078

(57) ABSTRACT

A stiffener for an endoscope may comprise a rod and a handle. The rod may be configured so that disposing the rod within the channel increases the stiffness of the endoscope shaft. The stiffener may comprise a channel for application of fluid there through. A kit may comprise a plurality of stiffeners of varying rigidity.

### CROSS-REFERENCE TO RELATED APPLICATIONS

This application is a continuation of U.S. patent application Ser. No. 14/293,779, entitled “METHOD AND APPARATUS FOR ENDOSCOPE STIFFENING, filed on Jun. 2, 2014. The above-listed application is commonly assigned with the present invention and is incorporated herein by reference as if reproduced herein in its entirety.

### BACKGROUND OF THE INVENTION

During insertion into and/or other movements of an endoscope within a body cavity, a scope shaft may be moved through a tortuous path. To enable this movement, the shaft of an endoscope may be at least partially flexible. However, with a flexible shaft an endoscope may sometimes become inadvertently deflected and move away from an intended direction. When force is placed against a deflected scope the distal end of the scope may be pressed against the walls of the patient's colon. Such force may be painful for the patient, and in some situations, distal parts of the colon may not be reached, or may only be reached with difficulty. To help alleviate this concern, and minimize discomfort and potential damage to body cavities, and to facilitate access to less accessible internal cavities, the diameter of modern endoscopes has tended to decrease.

While decreasing the diameter of an endoscope may be advantageous in some regards, small diameter endoscopes may be more flexible than many doctors prefer. For example, with increased flexibility the risk of scope buckling becomes higher, and it may be difficult to keep a modern endoscope from becoming slack, buckled, or twisted during insertion making insertion more difficult and time consuming. There remains a need for a convenient, low cost, and effective method and apparatus for more easily inserting and manipulating an endoscope by varying the stiffness thereof.

### BRIEF DESCRIPTION OF THE DRAWINGS

FIGS. 1A and 1B illustrate one embodiment of a stiffener for an endoscope.

FIGS. 2A and 2B illustrate another embodiment of a stiffener for an endoscope including an irrigation channel.

FIGS. 3A and 3B illustrate another embodiment of a stiffener for an endoscope that provides fluid connection through a handle of the stiffener.

FIG. 4 illustrates one embodiment of an endoscope stiffener kit.

FIG. 5A illustrates a stiffener inserted through a channel of an endoscope shaft.

FIG. 5B illustrates a stiffener inserted only partially through a channel of an endoscope shaft.

FIGS. 6A-G illustrate a top view (6A), a front view (6B), a right side view (6F), a left side view (6E), a back side view (6C), a bottom view (6D), and a perspective view (6G) of one embodiment of an endoscope stiffener.

FIGS. 7A-G further illustrate a top view (7A), a front view (7B), a right side view (7F), a left side view (7E), a back side view (7C), a bottom view (7D), and a perspective view (7G) of one embodiment of an endoscope stiffener.

FIGS. 8A-G further illustrate a top view (8A), a front view (8B), a right side view (8F), a left side view (8E), a back side view (8C), a bottom view (8D), and a perspective view (8G) of one embodiment of an endoscope stiffener.

FIG. 8H illustrates a handle and base of the embodiment of FIGS. 8A-G without a rod.

FIGS. 9A-G further illustrate a top view (9A), a front view (9B), a right side view (9F), a left side view (9E), a back side view (9C), a bottom view (9D), and a perspective view (9G) of one embodiment of an endoscope stiffener.

### DETAILED DESCRIPTION

The methods and apparatuses described herein relate to support or guide stiffeners configured to facilitate introduction of an endoscope into a body cavity. The stiffeners may be configured for insertion through existing channels of an endoscope, such as an endoscope treatment channel, and may provide support during scope insertion. For example, when inserted through a channel of an endoscope, the stiffener may increase flexural stiffness of the endoscope shaft, and help to prevent buckling or inadvertent twisting of the endoscope as it is navigated through a body cavity.

A stiffener may, in some embodiments, be partially or fully inserted through the length of an endoscope channel. For example, a user, such as a medical doctor, may be able to reversibly insert or remove the stiffener any distance through an endoscope channel. By changing the relative distance of insertion, a doctor may dynamically adjust relative proportions of a more flexible part of an endoscope and portions that are increased in flexural stiffness. Therefore, by moving the stiffener back and forth through an endoscope channel, the overall flexibility of an endoscope shaft may be readily and dynamically adjusted. The rigidity of a stiffener may be selected so that upon insertion of the stiffener through an endoscope the rigidity of the endoscope shaft may be increased but still enable the shaft to be flexible enough to traverse the path of the colon or other body cavity.

In some embodiments, a stiffener may be one part of a kit or group of stiffeners. And the relative ease with which the stiffener may be inserted and/or removed may make the stiffener particularly amenable to adjustment and may, for example, be accomplished by substituting one member of a kit with another member. For example, if a user finds it difficult to insert an endoscope because the stiffness of the scope is not suitable, the user may readily retract one stiffener and easily insert another stiffener having a different level of rigidity. Furthermore, some users may, for example, prefer to insert an endoscope at one stiffness level during one part of a procedure and then change the stiffness during another part of the procedure by, for example, removing a stiffener, or substituting a stiffener having a different level of rigidity. To encourage relative ease and reproducibility of insertion and retraction (e.g., to a known length), in some embodiments a stiffener may include suitable markings, e.g., hatch marks or graduations, which may be visible along the length of the support such that the extent of insertion of the stiffener may be readily made evident to a user.

For example, in some embodiments, a kit of stiffeners may include stiffeners that may vary in stiffness, length, coefficient of friction, or other properties and/or combinations of the aforementioned properties. A user may then be able to pick a particular stiffener as may be suitable for a particular patient, particular procedure or part of a procedure. Furthermore, in some embodiments, a user may choose one stiffener for a first part of a procedure, remove the stiffener, and then insert another stiffener for other stages of insertion or other parts of a procedure.

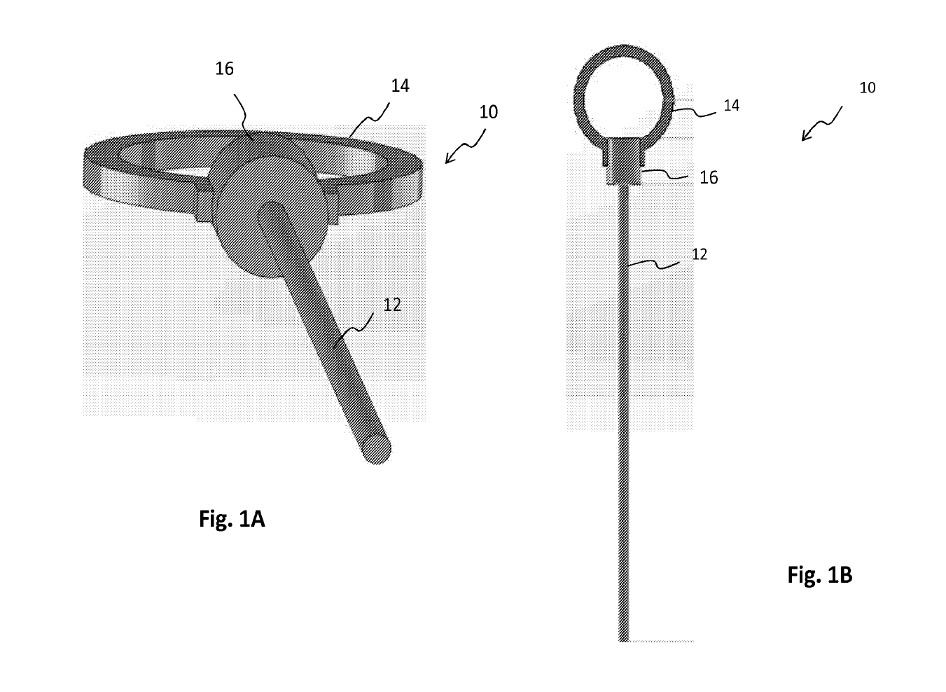

FIG. 1A shows a perspective view of an exemplary embodiment of an endoscope stiffener 10. FIG. 1B shows the same stiffener 10, but with the stiffener 10 oriented in a side elevational view. The stiffener 10 may be part of a kit or group of stiffeners or may be sold and/or used as a unitary device. The stiffener 10 may include a rod 12 configured for introduction through an endoscope channel. For example, a rod 12 of the stiffener 10 may, for example, be manually introduced through an available port of an endoscope. The rod 12 may have an outer diameter limited by the diameter of the channel into which it may be inserted. A suitable outer diameter may, for example, be within a range of about 1 mm to about 5 mm.

A stiffener 10 or the rod 12 of a stiffener 10 may, by way of non-limiting example, be made of any of various medical grade plastic materials. In some embodiments, the stiffener may be made from or include a polycarbonate, low density polyethylene (LDPE), or other suitable material. In some embodiments, at least some part of the rod 12 of the stiffener 10 may be coated with another material, such as polytetrafluoroethylene (PTFE). For example, a coating may be used to adjust or modify the surface frictional properties of the stiffener. Alternatively, the stiffener may include a rod 12 that includes texture as may be suited to adjust frictional contact between the rod 12 of stiffener 10 and the walls of an endoscope channel. For example, the rod 12 may be configured to slide relatively easily when a user applies an inserting force. In some embodiments, static frictional forces between the rod 12 and an endoscope channel wall may be great enough that when an endoscope channel is urged forward the stiffener 10 may also be urged forward. In some embodiments, the stiffener may comprise a stiffness that is greater than that of an endoscope shaft.

The rod 12 may be longer or shorter than a typical endoscope shaft. The rod 12 may be long enough to be useful in any of various colonoscopy procedures. And, a length of the rod 12 and relative position of handle 14, may, for example, and in some embodiments, be tailored for use in a particular procedure. For example, a rod 12 may, in some embodiments, be configured for use in either or both of a procedure for the upper or lower portions of the colon. For example, a rod that may be designed to access substantially all regions of a typical colon may be about 250 cm long. Of course, other lengths may be used for other endoscopy procedures and/or for some patients. A handle may have a T-shaped configuration, or a ring-shaped configuration, or comprise a plurality of rings.

A rod 12 may be solid or may be tubular. In some embodiments, the rod of a stiffener 10 may include one or more channels configured to receive a fluid such as may be used for suctioning or irrigation purposes. The stiffener 10 may further include a handle 14. The handle 14 may, for example, be configured and used to apply a force directing the rod 12 through an endoscope channel. For example, a handle 14 may be configured to allow a user to transfer a rotational and/or axial force to the rod 12. The stiffener 10 may further include a base 16. The base 16 may function to couple the rod 12 to the handle 14 and to provide a stop against further insertion. And, in some embodiments, the handle 14 and rod 12 may be a unitary device or the handle and rod may be removably connected.

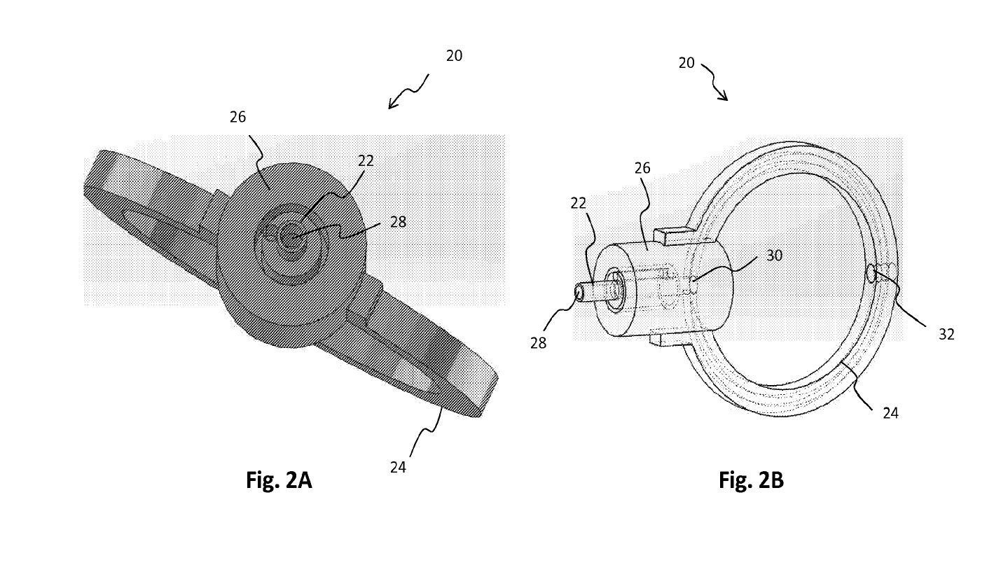

FIG. 2A shows another exemplary embodiment of an endoscope stiffener 20. FIG. 2B shows a same stiffener 20, but with the stiffener 20 oriented in a side perspective view. In the embodiment of stiffener 20, the rod 22 may be tubular and may include one or more channels 28 configured to carry fluid or allow insertion of tools down the treatment channel when the stiffener is inserted as may, for example, be desired to facilitate irrigation purposes. A base 26 may serve to link the handle 24 to the rod 22 of the stiffener 20 and may also, in some embodiments, include one portion of a fitting 30 as may be useful to seal or connect to applied fluid. For example, in some embodiments, the base 26 may include one part of a Luer-type connection fitting 30 and may be suited for connection to a source of irrigation fluid. The handle 24 may further include an opening 32 which may, for example, be convenient to route fluid tubing or another element towards the connection fitting 30. A handle 24 may be solid or may, in some embodiments, include a channel suitably configured to provide irrigation fluid. For example, a channel of the handle 24 may be configured to accept a fluid supply. Fluid may, in those embodiments, be directed through that channel and fluidly connected to a rod 22. Therefore, a hollowed portion of the handle 24 may be fluidly connected to rod 22, and the base 26 may be suitably configured to provide fluid connectivity substantially without fluid leakage.

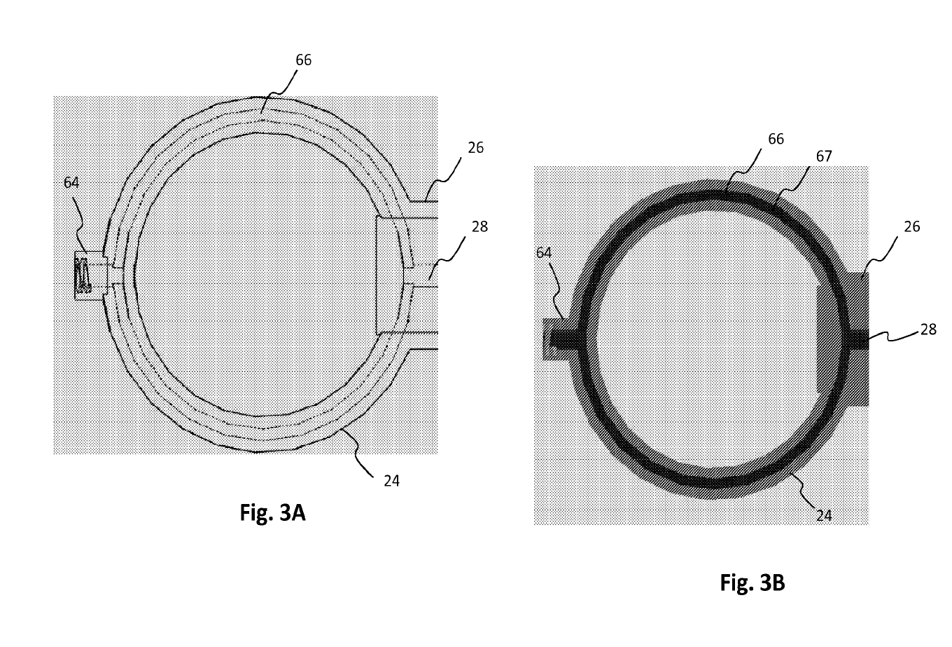

FIG. 3A illustrates an embodiment of an endoscope stiffener in which the handle 24 comprises a hollowed channel 66 and connection fitting 64. The connection fitting 64 may, for example, comprise a Luer-lock fitting, threads, a Luer-slip or slip-fit tapering, quarter-turn lock or other fitting suitable for removable connection to a fluid source. As shown in FIG. 3B, an irrigation fluid 67 may then be directed through the channel 66 towards base 26 and into a channel 28 of a rod.

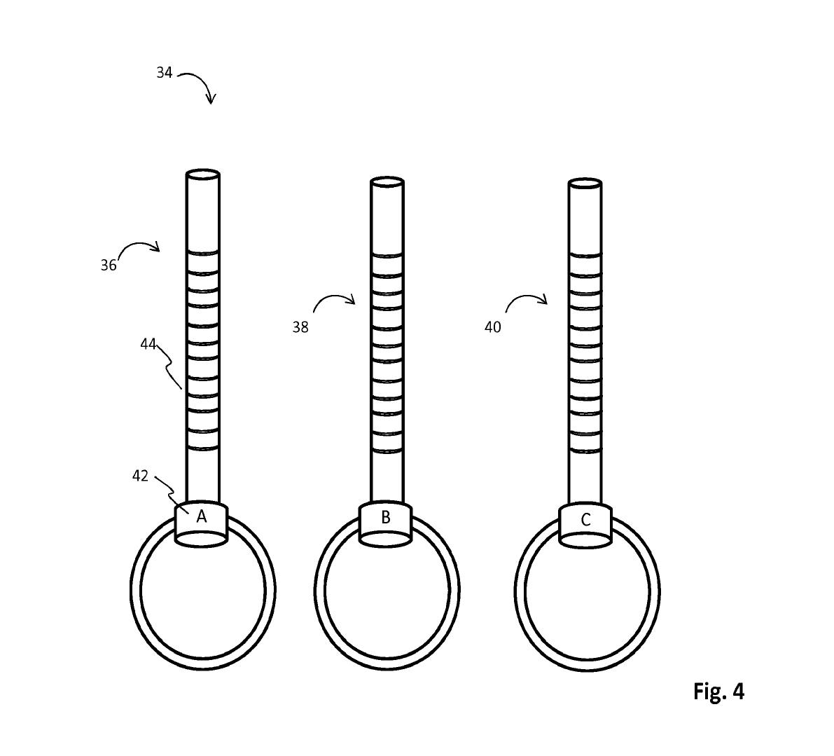

FIG. 4 shows an embodiment of a group of stiffeners 34 that may be included in a stiffener kit. The group 34 may include two or more individual stiffeners (36, 38, and 40). Those individual stiffeners may be graded in stiffness and labeled accordingly (42) so that a user may readily select a stiffener that has greater or lesser stiffness. As shown by the series of markings 44, in some embodiments a stiffener may include any number of markings to make evident to a user the distance in which the stiffener may be inserted.

In some embodiments, a kit of stiffeners may include stiffeners that may vary in stiffness and/or length. For example, an individual stiffener member among a stiffener kit may have a stiffness that is greater than another member of the kit. In some embodiments, varying stiffness among individual members of a kit may be achieved by providing individual members of slightly different diameters. In other embodiments, the diameter of different members of varying stiffness may be about the same. For example, the different members of a kit may have varying stiffness because the members are each constructed of a different material from the other stiffeners in the group. In some embodiments, members of a kit may include coated, uncoated and/or partially coated members, and for example, the presence or amount of a coating may be used to create stiffeners of a range of friction values.

An endoscope, in some embodiments, may be used for colonoscopy, which generally involves insertion of an endoscope shaft into the anus and maneuvering the shaft through the colon of the patient in order to perform any of various colonoscopy procedures. The stiffeners described herein may be suitably used for colonoscopy procedures. However, some stiffeners may be configured for other scopes, and may be inserted in other body cavities and used in other endoscopic procedures.

A user may employ a stiffener 10 to increase the stiffness of a flexible endoscope shaft, but still enable the doctor to navigate the stiffener through the curving path of any section of the colon or other bodily path. Increasing the stiffness of the endoscope shaft may be beneficial and enable a doctor to more easily advance the scope through, e.g., the colon, and to encourage advancement of the scope without buckling of the scope or formation of regions of endoscope slack. In practice, a user may, in some embodiments, extend the stiffener through the entirety of the endoscope channel or through only a portion of the endoscope channel. Therefore, an endoscope shaft may have a leading portion that is more or less rigid than trailing portions of the endoscope shaft.

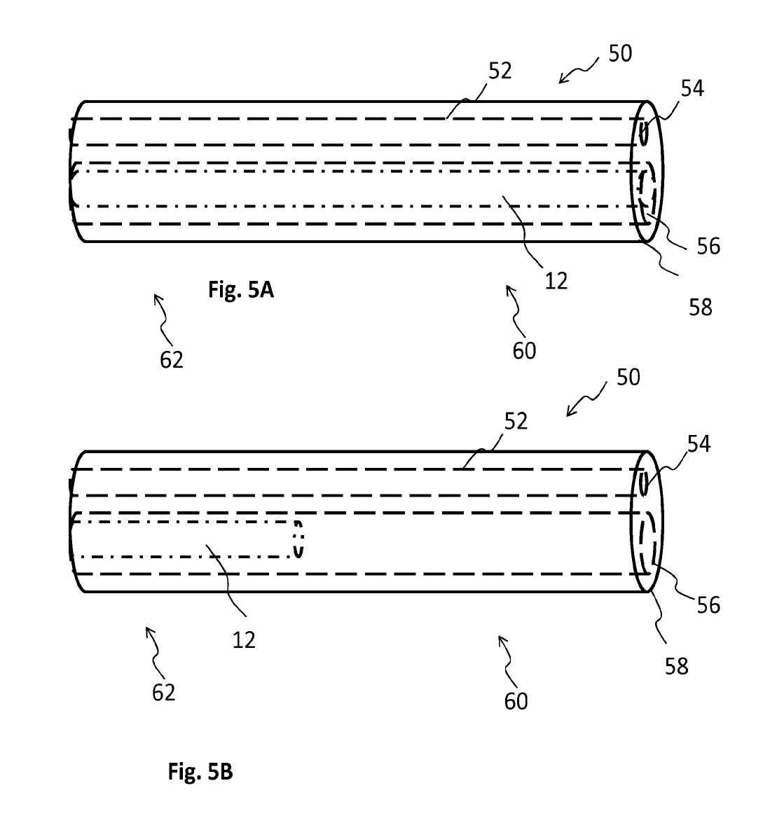

For example, FIG. 5A shows an endoscope shaft 50 that includes one channel 52 including an optical surface 54 as well as treatment channel 56. As shown in FIG. 5A, rod 12 (as shown using the dot-dashed line) is shown as approximately fully inserted through the treatment channel 56 of shaft 50 at or near the end 58 of the endoscope shaft. Thus, the rigidity of the leading portion or tip 60 of the endoscope shaft 50 may therefore be increased. As shown in the FIG. 5B, rod 12 is only partially extended through the treatment channel 56 of endoscope shaft 50 and a leading portion 60 of the endoscope may not be as rigid as trailing portion 62. In the event that a leading edge portion 60 of the stiffener becomes kinked or inadvertently bent, a doctor may retract the endoscope from the body and along the rod 12. As the endoscope tip 60 is pulled back and over the rod 12, the endoscope shaft may be realigned and any kinks or bends in the endoscope shaft 50 may be removed or made less severe. A user may therefore, in some embodiments, manipulate the positioning of both the endoscope shaft 50 and/or the stiffener 10 to encourage proper positioning of an endoscope through the colon.

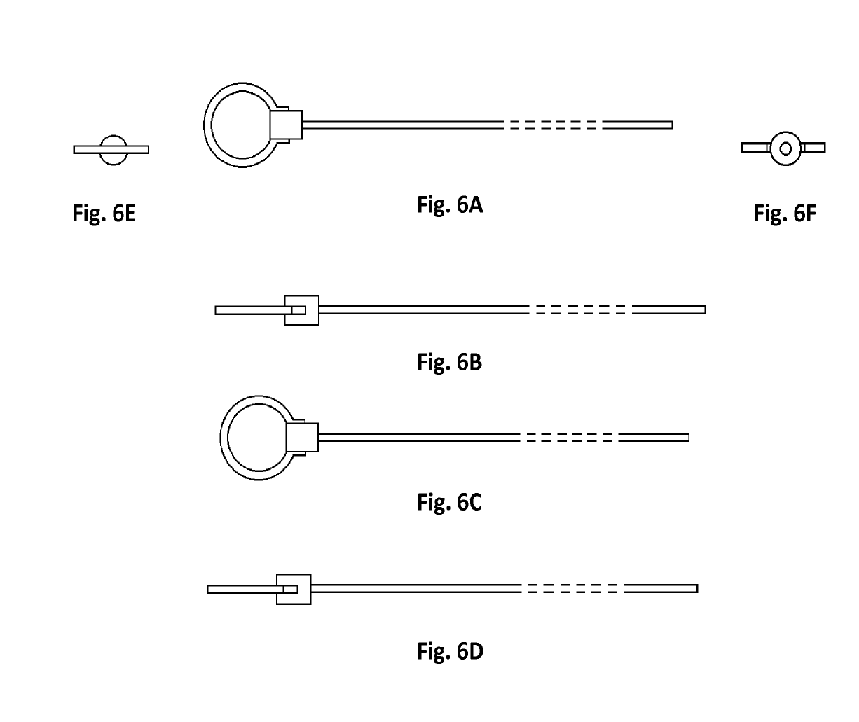

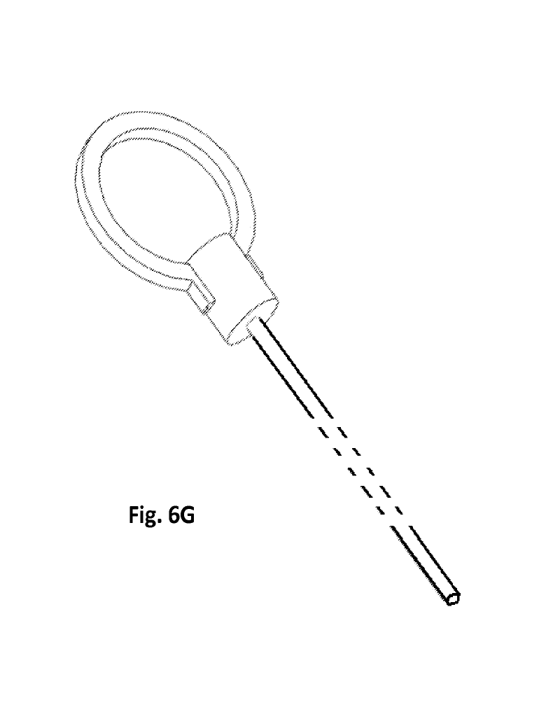

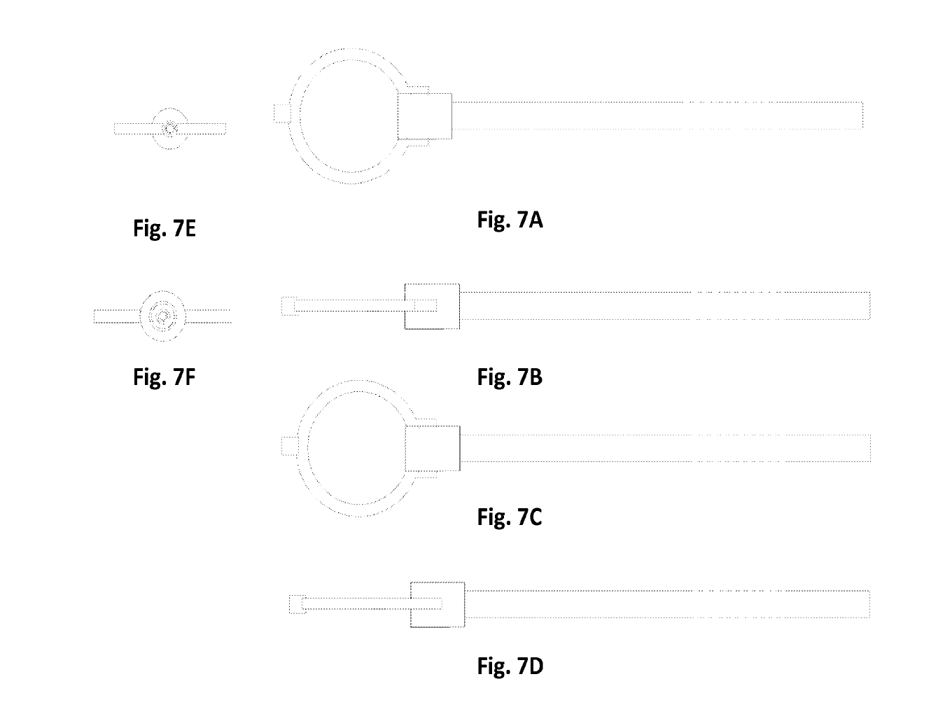

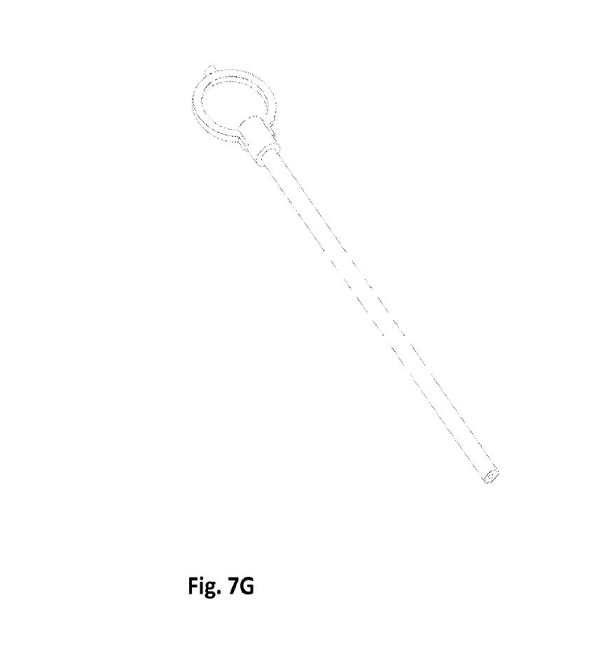

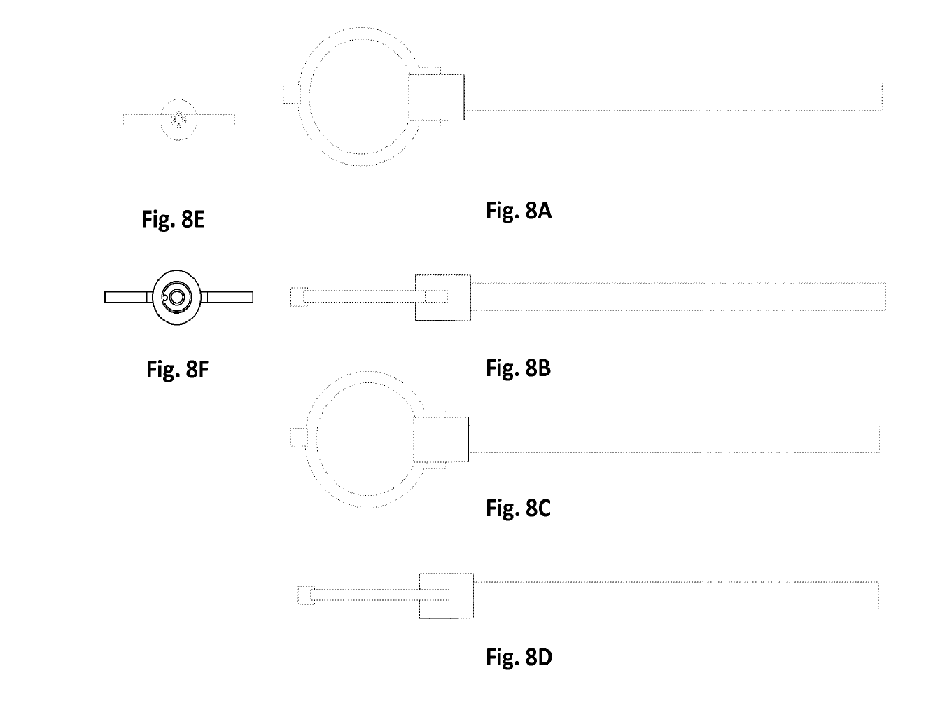

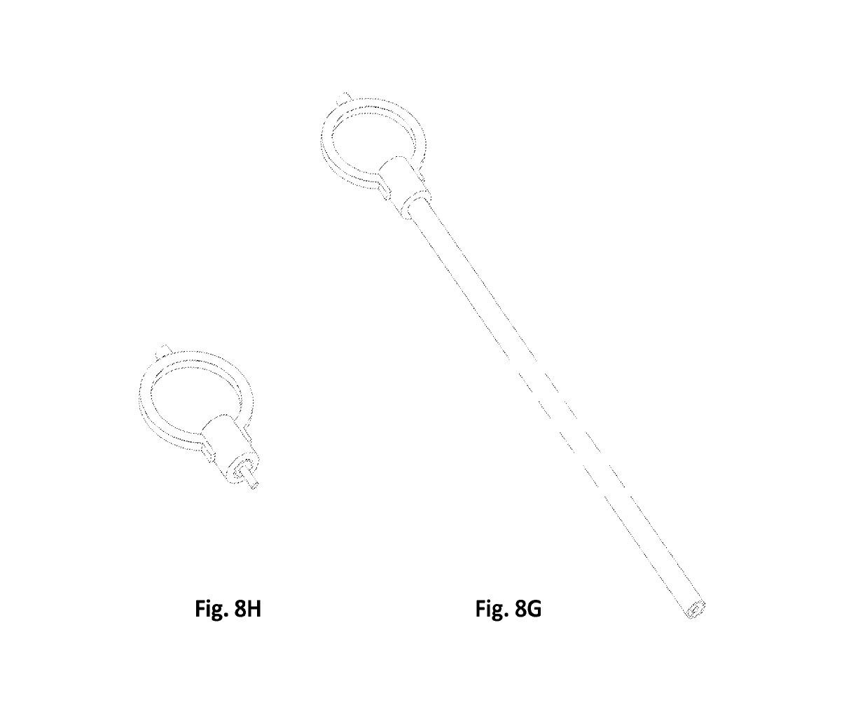

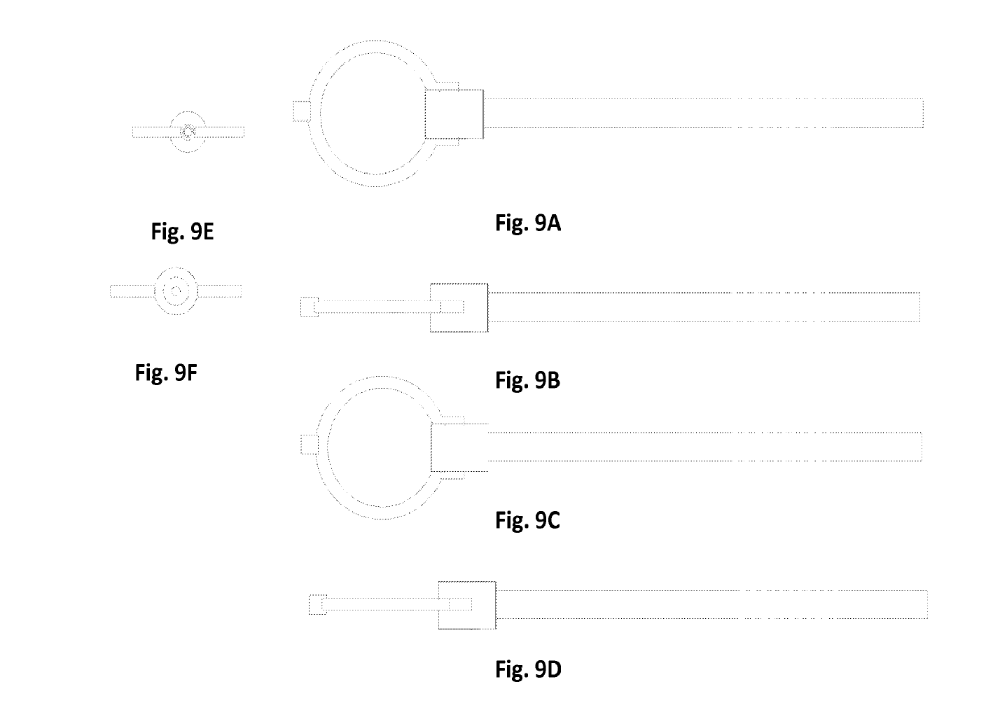

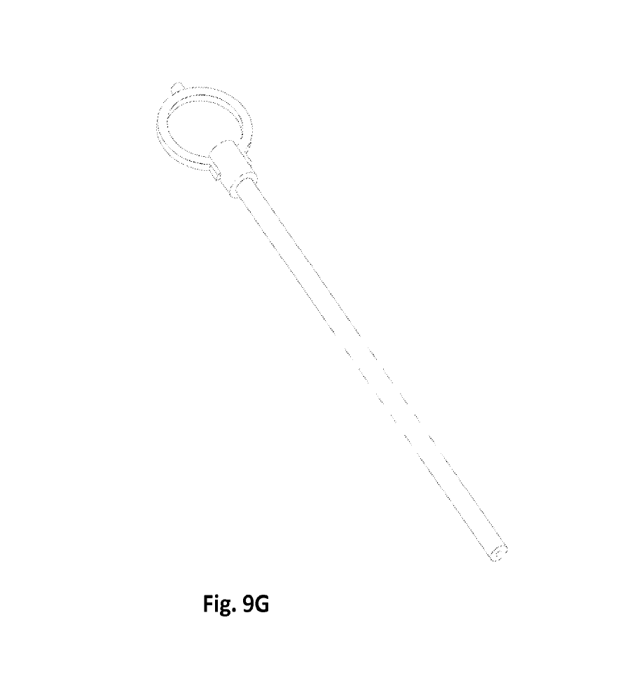

FIGS. 6A-G, 7A-G, 8A-H and 9A-G further illustrate various embodiments of a stiffener in various views. The purpose of the broken lines in those figures is to indicate that the length of the rod may vary, it being understood that the rod has a uniform shape and appearance throughout its length.

Although the disclosed subject matter and its advantages have been described in detail, it should be understood that various changes, substitutions and alterations can be made herein without departing from the invention as defined by the appended claims. Moreover, the scope of the claimed subject matter is not intended to be limited to the particular embodiments of the process, machine, manufacture, composition, or matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

What is claimed is:

1. An apparatus for supporting an endoscope shaft comprising:
a handle; and
a rod connected to the handle and configured for disposition within a channel of the endoscope shaft, wherein the rod is configured so that disposing the rod within the channel increases the stiffness of the endoscope shaft, and comprises a first working channel configured to receive an irrigation fluid or endoscope tool.

2. The apparatus of claim 1 wherein the handle and rod comprise a unitary piece.

3. The apparatus of claim 1 wherein the handle and rod are removably connectable.

4. The apparatus of claim 1, wherein the handle comprises a second working channel in fluid communication with the first working channel, the handle further comprising a fitting suitable for sealed connection of the second working channel to a fluid source.

5. The apparatus of claim 5, the fitting comprising a Luer-type fitting.

6. The apparatus of claim 1, the rod further comprising graduations.

7. The apparatus of claim 1, wherein the rod is configured for insertion into a treatment channel of an endoscope.
